(19) United States
(12) Patent Application Publication
(10) Pub. No.: US 2019/0282489 A1
(43) Pub. Date: Sep. 19, 2019

(54) SOLID COMPOSITION COMPRISING A POWDER AND A POLY(ALKYLENE OXIDE)

(71) Applicant: L'Oreal, Paris (FR)

(72) Inventors: Ségolène DE MENTHIERE, Saint Ouen (FR); Gérard GABIN, Saint Ouen (FR); Rafik KERBOUSSA, Saint Ouen (FR)

(21) Appl. No.: 16/463,923

(22) PCT Filed: Nov. 27, 2017
(86) PCT No.: PCT/EP2017/080528

§ 371 Date: May 24, 2019

(30) Foreign Application Priority Data
Nov. 28, 2016 (FR) 1661589

(51) Int. Cl. A61K 8/86; A61Q 5/06

(52) U.S. Cl. CPC A61K 8/86; A61Q 5/06

(57) ABSTRACT

The invention relates to a solid composition comprising (i) one or more powders, and (ii) one or more poly(alkylene oxides), and to the use thereof for styling the hair.

The present invention relates to a solid composition comprising at least one powder and at least one poly(alkylene oxide), to the process for manufacturing same, to the use thereof for shaping keratin fibres and to a cosmetic treatment process using this composition.

In the field of the cosmetic treatment of keratin fibres, especially human keratin fibres, such as the hair, and more particularly in the field of hairstyling and haircare, styling powders have existed for a number of years, either in loose powder form or in aerosol form. By depositing on the surface of the hair, they create roughnesses and make it possible to obtain lifting of the roots and volume, and to give the head of hair texture and body and also repositionable hold rapidly without wetting the head of hair.

The powders used may be of mineral, organic or synthetic origin, but are usually of mineral nature.

In practice, loose powders dispensed in powder shakers or in aerosols are not entirely satisfactory and may cause discomfort as a result of their volatile and nanometric nature. They have a tendency to make the hair very matt and are more difficult to remove on shampooing.

There is thus a need to develop novel compositions that can overcome the abovementioned drawbacks.

The Applicant has now discovered that a combination of at least one powder with at least one poly(alkylene oxide) as binder makes it possible to afford improved working qualities especially such as ease of handling, reduced volatility, low friability, in particular when the composition according to the invention is in compacted powder form, and styling properties such as providing the head of hair with volume and mass, and lifting of the roots, while at the same time being able to be removed easily on shampooing.

In addition, the composition makes it possible to obtain a less pronounced matt effect than that of the products used hitherto.

One subject of the invention is thus a solid composition comprising:

(i) one or more powders, and
(ii) one or more poly(alkylene oxides).

The term “solid composition” denotes a composition which has a hardness such that it does not flow under its own weight, as opposed to compositions that are “fluid”, at room temperature (25° C.) and at atmospheric pressure (760 mmHg).

This composition is preferably in a compacted form.

A subject of the present invention is also a process for preparing this composition, preferably in the form of a compacted powder.

A compacted powder more particularly denotes a powder pressed using a manual or mechanical press.

Another subject of the invention consists of a cosmetic process for treating keratin fibres, especially human keratin fibres, such as the hair, and more particularly for shaping and/or conditioning the hair, comprising the use of the composition according to the invention.

The invention also relates to the use of a composition according to the invention for shaping and/or conditioning keratin fibres, especially human keratin fibres, such as the hair.

Other subjects, characteristics, aspects and advantages of the invention will emerge even more clearly on reading the description and the example that follows.

In the text hereinbelow, and unless otherwise indicated, the limits of a range of values are included in that range, in particular in the expressions “between” and “ranging from . . . to . . . ”.

The expression “at least one” used in the present description is equivalent to the expression “one or more”.

According to the invention, the solid composition comprises:

(i) one or more powders, and
(ii) one or more poly(alkylene oxides).

The composition according to the invention comprises, as first essential ingredient, one or more powders.

Preferably, the powder may be a styling powder.

For the purposes of the present invention, the term “styling powder” means a powder which has a capacity for shaping the head of hair or for the durability of this shaping.

The capacity for shaping or shaping durability of the powder may especially be due to its chemical nature and/or its geometrical form and/or its arrangement configuration during deposition onto the keratin fibre. Specifically, the irregularities created at the surface of the hair promote the inter-attachment of the fibres.

The powder may be of any form such as lamellar, spherical or oblong, irrespective of the crystallographic form (for example cubic, hexagonal, orthorhombic, rhombohedric or tetragonal). In a preferred embodiment, the powders are not spherical.

The number-average size of the powder may range from 0.001 to 50 μm, better still from 0.002 to 40 μm and even more preferentially from 0.003 to 35 μm.

This number-average size corresponds to the size measured from the statistical distribution of the particle sizes for half of the total number of the particles. This size is referred to as the D50.

In addition, the number-average size of these particles may be measured in the form of a mean value via an observation method with a light microscope, an electronic microscope, or a particle size analyser using laser scattering.

In the case where the particles are not in spherical form, their number-average size may be determined in the form of the mean of the longest or shortest diameter or of the thickness.

Said powder may be mineral or organic, preferably mineral. It may especially be a filler, a pigment or a mixture thereof. Preferably, the powder is a filler.

For the purposes of the present invention, the term “filler” means natural or synthetic, white or colourless particles of any form, which are insoluble in the medium of the composition, irrespective of the temperature at which the composition is manufactured.

The fillers may be organic or inorganic, and may be of any form such as lamellar, spherical or oblong, irrespective of the crystallographic form (for example cubic, hexagonal, orthorhombic, rhombohedric or tetragonal). In a preferred embodiment, the fillers are not spherical.

For the purposes of the present invention, the term “pigment” means organic or mineral, white or coloured particles of any form, which are insoluble in the medium of the composition and which give the composition a colour.

The term “mineral” encompasses natural or synthetic chemical compounds that are inorganic. Mineral substances are mainly in a crystalline form.

Examples of mineral or inorganic powders that may especially be mentioned include:
fillers such as metal carbonates, oxides and sulfates such as those of alkaline-earth metals, aluminium, gallium and indium; silicates; modified or unmodified silicas; sericite, synthetic fluorophlogopite, talc; natural or synthetic mica, especially white mica, gold mica, red mica, black mica and mica-lithium oxide; calcium phosphate, silicic acid, silicic anhydride, silicon carbide, metal salts of tungstic acid, magnesium aluminate, bentonite, zeolites, smectite, hydroxyapatite, ceramic powder, boron nitride and glass or ceramic microcapsules;
specific composite fillers such as those sold under the names Excel Mica, Excel Pearl and the powder La Vie by the company Miyoshi Kasei, Inc.;
white pigments such as titanium dioxide, zinc oxide, zirconium oxide and cerium oxide;
coloured pigments such as red iron oxide, yellow iron oxide, black iron oxide, chromium oxide, chromium hydroxide, Prussian blue, ultramarine blue, chromium hydrate, ferric blue, inorganic blue pigments, carbon black, lower titanium oxides, manganese violet, cobalt violet, and metal powders such as aluminium powder and copper powder;
nacreous pigments such as bismuth oxychloride, mica/titanium, essence of pearl, powder prepared by coating synthetic mica with titanium dioxide, powder prepared by coating silica flakes with titanium dioxide, which is available under the brand name Metashine from Nippon Sheet Glass Co., Ltd, powder prepared by coating alumina flakes with tin oxide and titanium dioxide, powder prepared by coating aluminium flakes with titanium dioxide, powder prepared by coating copper flakes with silica, sold by the company Eckert Inc. USA, powder prepared by coating bronze flakes with silica, and powder prepared by coating aluminium flakes with silica;
ultrafine powder, having a mean particle size of less than 0.1 μm, such as ultrafine titanium dioxide, ultrafine zinc oxide, ultrafine iron oxide, and ultrafine cerium oxide;
other powders such as the luminescent powder sold under the brand name Luminova Series by Mitsui & Co., Ltd., aluminium powder, stainless-steel powder, tourmaline powder, and amber powder; and
a mixture thereof.

Examples of organic powders are wool powder, polyamide powder (Nylon® or Orgasol® from Arkema), polyester powder, polyethylene powder, polypropylene powder, polystyrene powder, polyurethane powder, benzoguanamine powder, polymethyl benzoguanamine powder, tetrafluoroethylene powder, poly(methyl methacrylate) powder, cellulose powder, silk powder, silicone powder, silicone rubber powder, powders of synthetic resins such as a styrene/acrylate copolymer, a divinylbenzene/styrene copolymer, a vinyl resin, a urea resin, a phenolic resin, a fluoro resin, tetrafluoroethylene (Teflon®) polymers, a silicone resin, an acrylic resin, a melamine resin, an epoxy resin, and a polycarbonate resin, hollow polymer microspheres, such as those of poly(vinylidene chloride)/acrylonitrile, for example Expancel® (Nobel Industrie), or acrylic acid copolymers (Polytrap® from the company Dow Corning), silicone resin microbeads (for example Tospearls® from Toshiba), particles formed from polyorganosiloxane elastomers, microcrystalline fibre powder, starch powder, acylated lysine powder, poly-β-alanine, lauryllysine, powder of the metal salt of (long-chain alkyl) phosphate, metal soap powder, Colour Index (CI) yellow pigments, CI orange pigments and tar-based pigments prepared in lacquer form, and dyes existing in the natural state prepared in lacquer form.

The tar-based dyes include, for example, the dyes Red No. 3, Red No. 10, Red No. 106, Red No. 201, Red No. 202, Red No. 204, Red No. 205, Red No. 220, Red No. 226, Red No. 227, Red No. 228, Red No. 230, Red No. 401, Red No. 505, Yellow No. 4, Yellow No. 5, Yellow No. 202, Yellow No. 203, Yellow No. 204, Yellow No. 401, Blue No. 1, Blue No. 2, Blue No. 201, Blue No. 404, Green No. 3, Green No. 201, Green No. 204, Green No. 205, Orange No. 201, Orange No. 203, Orange No. 204, Orange No. 206, and Orange No. 207.

The natural dyes include powders such as carmine, laccaic acid, carsamine, brazilin and crocin.

The doped or undoped composite powder may also be suitable as base powder intended to undergo a surface treatment. Examples of the latter include the powder prepared by coating inorganic dye pigments such as red iron oxide with silicic anhydride, powders prepared by coating Nylon with white pigments and powders prepared by coating fillers with ultrafine white pigments.

The mineral powder(s) according to the invention may optionally be surface-modified with organic compounds.

More preferentially, the powder is a mineral powder constituted of one or more water-insoluble mineral compounds.

For the purposes of the present invention, the term “water-insoluble” refers to a compound whose solubility at spontaneous pH in water at 25° C. and at atmospheric pressure is less than 0.1%.

The water-insoluble mineral compound(s) are preferably chosen from metal carbonates, oxides and sulfates, silicates, modified or unmodified silicas, mica, talc, and mixtures thereof.

Examples that may more particularly be mentioned include the carbonates, oxides and sulfates of alkaline-earth metals such as beryllium, magnesium, calcium, strontium, barium and radium, better still magnesium and calcium; the oxides, sulfates and carbonates of aluminium, gallium and indium; silicates such as kaolinite or kaolins (natural silicates containing kaolinite), silicates containing magnesium, particularly those containing an amount of magnesium of greater than 10% by weight (on a dry basis) expressed as magnesium oxide, such as Li—Mg—Na silicates, for instance Laponite XLG, proposed by the company Rockwood; modified or unmodified silicas, better still modified silicas; mica; talc; and mixtures thereof.

Among the modified silicas, it is preferred to use surface-treated silicas such as hydrophobic silicas, for instance hydrophobic fumed silica of nanometric size and surface-treated with hexamethyldisilazane, such as the silica sold under the trade name Aerosil R812S or Aerosil R972 by the company Evonik, or HDK H115 by the company Wacker, or the hydrophobic fumed silica surface-treated with dimethylsilane.

More preferentially, the powder(s) are chosen from calcium carbonate, magnesium carbonate, alumina, barium sulfate, magnesium oxide, kaolinite or kaolins, modified or unmodified silicas, better still modified silicas and even better still hydrophobic fumed silica of nanometric size and surface-treated with hexamethyldisilazane or hydrophobic fumed silica surface-treated with dimethylsilane; mica; talc; and mixtures thereof The hydrophobic silica is especially chosen from silicas with a specific surface area of from 50 to 500 m²/g, a number-average particle size ranging from 3 to 50 nm and a packed density ranging from 40 to 200 and better still from 50 to 150 g/l.

The powder(s) are preferably present in an amount of greater than or equal to 30% by weight, better still greater than or equal to 40% by weight, even better still greater than or equal to 50% by weight, even more preferentially greater than or equal to 60% by weight, relative to the total weight of the solid composition.

More particularly, the powder(s) are present in a total amount ranging from 30 to 99% by weight, better still from 40 to 95% by weight, even more preferentially from 50 to 93% by weight, better still from 60 to 90% by weight, relative to the total weight of the solid composition.

The composition according to the invention comprises, as second essential ingredient, one or more poly(alkylene oxides).

The poly(alkylene oxides) used in the invention may be homopolymers of C2-C4 and better still C2-C3 alkylene oxide, or copolymers of at least two C2-C4 and better still C2-C3 alkylene oxides. Examples of alkylene oxides that may especially be mentioned include ethylene oxide, propylene oxide and butylene oxide. Preferably, the alkylene oxide units are ethylene oxide units.

Preferably, the poly(alkylene oxide(s)) used in the present invention are chosen from polyethylene glycols comprising from 2 to 100 ethylene oxide (or EO) units, better still from 3 to 50 EO and even better still from 5 to 10 EO.

Preferably, the poly(alkylene oxides) are present in an amount ranging from 0.1 to 30% by weight, more preferentially from 1 to 20% by weight and better still from 5 to 15% by weight, relative to the total weight of the solid composition.

The powder(s) and the poly(alkylene oxide(s)) are preferably present in a powder(s)/poly(alkylene oxide(s)) weight ratio ranging from 1 to 50, better still from 4 to 30 and even more preferentially from 5 to 20.

The solid compositions according to the invention may also contain one or more additives chosen from anionic, amphoteric or zwitterionic polymers, nonionic polymers, cationic polymers, nonionic, anionic, cationic, amphoteric or zwitterionic surfactants, ceramides, pseudoceramides, vitamins and provitamins including panthenol, water-soluble and liposoluble sunscreens, nacreous agents, opacifiers, sequestrants, solubilizers, antioxidants, antidandruff agents, anti-seborrhoeic agents, agents for preventing hair loss and/or for promoting hair growth, penetrants, fragrances, peptizers and preserving agents, or any other additive conventionally used in the cosmetics field.

These additives may be present in the composition according to the invention in an amount ranging from 0 to 20% by weight relative to the total weight of the composition.

A person skilled in the art will take care to select these optional additives and the amounts thereof so that they do not harm the properties of the compositions of the present invention.

The compositions according to the invention may be in compacted powder form. They may be used as styling products.

The solid composition according to the invention may be manufactured via a known process. Mention may be made especially of the process that consists in compression-moulding the solid composition as described above.

The compression-moulding process for manufacturing a composition according to the invention comprises the following steps consisting in:
(1) mixing the powder(s) with the poly(alkylene oxide(s)) with stirring, at room temperature (18-25° C.) and at atmospheric pressure,
(2) optionally milling and screening the mixture obtained in the first step,
(3) transferring the mixture into a container, such as a metal dish, and
(4) compression-moulding at a pressure ranging from 0.5 to 10 MPa and preferably from 1 to 5 MPa.

In step (1), any type of apparatus may be used. Examples of apparatus that may especially be mentioned include a Henschel mixer, a blending mixer, a V-shaped mixer, a band mixer, a Nauta mixer and a high-speed mixer.

In step (2), the optional milling may take place using milling apparatus such as a hammer mill, a pin mill or an atomizer. This milling is then followed by passing the milled material obtained through a screen. In addition, it is possible to apply vibrations for the purpose of increasing the homogeneity of the mixture.

In step (4), a hydraulic or electric compactor may be used.

In particular, the electric compactor Vetraco CP012 allows better control of the compaction parameters such as the pressure, the pressure-maintenance time, the filling of the dish and the pressure-increase rate.

The container, such as the metal dish, is placed in a powder-filled tool in which slides a punch which compacts the powder at the desired pressure. Various shapes and sizes of dishes exist according to the sales packaging. The weight of powder and the pressure are to be optimized in the dish selected.

The finished product is evaluated according to the following characteristics:
the disintegration, which is the capacity of a compacted composition to release the amount of powder required for use. It consists in rubbing the surface of the product, using the applicator specified for marketing, or with a finger if the product is intended to be used in this way. If the product is too hard, it will be difficult to disintegrate. In contrast, if the product is not hard enough, it will disintegrate too easily;
the hardness, which represents the resistance to penetration into a compacted composition. The higher the hardness, the more difficult the disintegration;
the mechanical strength, which is assessed by means of the drop test. These drops are performed manually: the dishes are released 10 times from a height of 30 cm onto a calibrated hard, flat surface. An average is determined for six dishes. The maximum tolerated percentage loss is 10%.

This process for preparing the compacted powder makes it possible to obtain a compact presentation form of powder that is less volatile, which has a styling effect. This is reflected especially by a provision of volume and lifting of the roots. Moreover, this composition makes it possible to reduce the matt effect and it is easier to remove with shampooing than the existing products, for instance the product sold under the brand name Carré d'Or from Piacelabo.

The present invention also relates to a process for cosmetic treatment, and more particularly for shaping, of keratin fibres, especially human keratin fibres and in particular the hair, which comprises the application to said fibres of an effective amount of a solid composition as described above.

This application is not rinsed out.

The present invention also relates to the use of the solid composition according to the invention for shaping the hair.

The invention is illustrated by the example that follows.

### EXAMPLE

A composition according to the invention was prepared from the ingredients indicated in the table below. The amounts are indicated as weight percentages of starting material in unmodified form relative to the total weight of the composition.

| | |
|---|---|
| Kaolinite¹ (powder) | 26.90 |
| Hydrophobic fumed silica treated with | 11.92 |
| hexamethyldisilazane² (powder) | |
| Hydrophobic fumed silica surface-treated with | 11.92 |
| dimethylsilane (powder) | |
| Mica (powder with a particle size of from 5 to 11 μm) | 22.41 |
| Talc | 11.95 |
| Magnesium stearate³ (powder) | 2.00 |
| Polyethylene glycol (8 EO) | 5.55 |
| Poly(dimethyldiallylammonium chloride) in water at | 5.55 |
| 40% of AM, non-stabilized | |
| 2-Phenoxyethanol | 1.00 |
| Fragrance | 0.30 |
| 1,2-Octanediol | 0.50 |

AM: Active material
¹Kaolin Supreme from Imerys
²Aerosil R812S - Evonik
³Magnesium stearate - Stéarineries Dubois

This composition was prepared by:
mixing the powders and the solid starting materials in a mixer equipped with paddles, a lump breaker and a jacket, so as to obtain a product that is as homogeneous as possible,
incorporating the liquid starting materials, by spraying using a spraying nozzle,
manual or automatic screening to make the powder more uniform and to break up any agglomerates before compacting, the screen used having a mesh size of 250 μm, and
compacting in metal dishes using an electric compactor, at a pressure expressed as force of between, for example, 500 and 1200 kg.

The powder thus compacted is applied onto the hair by finger or using a suitable applicator.

A soft, homogeneous, smooth feel is then obtained, along with a provision of volume and mass and lifting of the roots.

1-12. (canceled)

13. A solid composition comprising:
(i) at least one powder, and
(ii) at least one poly(alkylene oxide).

14. The solid composition according to claim 13, wherein the at least one powder comprises at least one water-insoluble mineral compound chosen from metal carbonates, metal oxides, metal sulfates, silicates, modified silicas, unmodified silicas, mica, talc, or mixtures thereof.

15. The solid composition according to claim 13, wherein the at least one powder is chosen from calcium carbonate, magnesium carbonate, alumina, barium sulfate, magnesium oxide, kaolinite or kaolins, modified silicas, unmodified silicas, mica, talc, or mixtures thereof.

16. The solid composition according to claim 13, wherein the at least one powder is chosen from modified silicas, mica, talc, or mixtures thereof.

17. The solid composition according to claim 13, wherein the at least one powder is present in an amount greater than or equal to 30% by weight, relative to the total weight of the solid composition.

18. The solid composition according to claim 13, wherein the at least one powder is present in an amount greater than or equal to 60% by weight, relative to the total weight of the solid composition.

19. The solid composition according to claim 13, wherein the total amount of the at least one powder ranges from 30% to 99% by weight, relative to the total weight of the solid composition.

20. The solid composition according to claim 13, wherein the total amount of the at least one powder ranges from 50% to 93%, relative to the total weight of the solid composition.

21. The solid composition according to claim 13, wherein the at least one poly(alkylene oxide) is chosen from polyethylene glycols comprising from 2 to 100 ethylene oxide (EO) units.

22. The solid composition according to claim 13, wherein the at least one poly(alkylene oxide) is chosen from polyethylene glycols comprising from 5 to 10 ethylene oxide (EO) units.

23. The solid composition according to claim 13, wherein the at least one poly(alkylene oxide) is present in an amount ranging from 0.1% to 30% by weight, relative to the total weight of the solid composition.

24. The solid composition according to claim 13, wherein the at least one poly(alkylene oxide) is present in an amount ranging from 5% to 15% by weight, relative to the total weight of the solid composition.

25. The solid composition according to claim 13, wherein the at least one powder and the at least one poly(alkylene oxide) are present in the solid composition at a powder/poly(alkylene oxide) weight ratio ranging from 1 to 50.

26. The solid composition according to claim 13, wherein the at least one powder and the at least one poly(alkylene oxide) are present in the solid composition at a powder/poly(alkylene oxide) weight ratio ranging from 5 to 20.

27. The solid composition according to claim 13, wherein the composition is in the form of a compacted powder.

28. A process for manufacturing a solid composition comprising at least one powder and at least one poly(alkylene oxide), comprising the following steps:
(1) mixing the at least one powder with the at least one poly(alkylene oxide) with stirring;
(2) optionally milling and screening the mixture obtained in the first step;
(3) transferring the mixture into a container; and
(4) compression-molding at a pressure ranging from 0.5 to 10 MPa.

29. A cosmetic process for treating keratin fibers, comprising applying to said fibers an effective amount of a solid composition comprising at least one powder and at least one poly(alkylene oxide).
